(19) United States
(12) Patent Application Publication
(10) Pub. No.: US 2024/0216001 A1
(43) Pub. Date: Jul. 4, 2024

(54) ERGONOMIC SURGICAL INSTRUMENTS

(71) Applicant: University of Massachusetts, Boston, MA (US)

(72) Inventor: Raymond Dunn, Shrewsbury, MA (US)

(21) Appl. No.: 18/288,786

(22) PCT Filed: Apr. 29, 2022
(86) PCT No.: PCT/US2022/027137

§ 371 Date: Oct. 27, 2023

Related U.S. Application Data
(60) Provisional application No. 63/181,849, filed on Apr. 29, 2021.

(51) Int. Cl. A61B 17/30; A61B 17/3211; A61C 1/10; A61B 17/00

(52) U.S. Cl. CPC A61B 17/30; A61B 17/3211; A61C 1/10; A61B 2017/00424; A61B 2017/00433

(57) ABSTRACT

Contoured surgical instruments, including contoured forceps gripping surfaces, are presented. The instruments can have a first arm with a contoured surface and a second arm with a contoured surface to provide for rotation in the hand of a user. Instruments including the contoured gripping surfaces have an ergonomic shape, favorable weight distribution, improved tactile response, and a construction suitable for precise grasping and manipulation required in many surgical procedures. The contoured gripping surfaces are applicable to a variety of dental and surgical instruments.

### CROSS REFERENCE TO RELATED APPLICATIONS

This application claims the benefit of priority to U.S. Provisional Application No. 63/181,849, filed Apr. 29, 2021, the entire contents of this application being incorporated by reference herein.

### BACKGROUND OF THE INVENTION

Surgical forceps have been in wide use to provide for grasping and precise manipulation of objects or tissue in proximity to surgical sites. Surgeons frequently use forceps to hold and manipulate tissue with their secondary hand during procedures while their primary hand is used to perform a procedure such as cutting or suturing. This process can be prolonged, frequently lasting many hours, resulting in fatigue. Surgeons performing hundreds of procedures over many years can experience arthritic conditions due to the repetitive manipulation of instruments during these procedures. Frequently wounds or incisions require movement within confined spaces and the combination of fatigue and the need for precision can adversely impact surgical outcomes. Frequently, the surgeon must rotate his entire arm at the shoulder, as the flat forceps handle that is frequently used in operating rooms cannot be rotated easily without risk of damaging tissue. There is also a need for sterility and cost effective solutions in medical care so that surgical instruments can be repeatedly used and sterilized if possible without loss of performance. Thus, an ongoing need exists for improvements in surgical implements to both lower cost and improve the quality of care.

### SUMMARY OF THE INVENTION

The present invention relates to handheld surgical instruments having an ergonomic shape, weight distribution, tactile response and construction suitable for precise grasping and manipulation that is required in many surgical procedures. Preferred embodiments utilize textured flexible materials for the grasping surfaces where the hands and fingers of the user grip the instruments to improve friction and provide the user with tactile feedback indicating the level of force being applied to tissue being manipulated by the instrument. A preferred embodiment of the invention uses contoured arms of surgical forceps having generally rounded handle surfaces along the longitudinal length of both arms. The proximal ends of both forceps arms are joined at one end of the forceps such that both arms flex with a spring characteristic that defines the force needed to close the forceps and also open the forceps with a reduction in applied pressure by the user's fingers. This shape and texture of the grasping surface area enables the surgeon to grasp and easily rotate the forceps to different angles while wearing latex gloves to reduce the need to hold the handle differently with a different grip. This enables rotation of the forceps within a body cavity without a substantial change of arm position and viewing angle. The contoured and textured surface provides an increase in the grasped surface area thereby increasing the amount of frictional contact at many angles. The textured surface has raised flexible elements periodically spaced across the contoured surface that compress while being gripped. The compressibility of the raised flexible elements on surface that compress upon grasping by the user provides haptic feedback to the user thereby allowing the user to adjust the level of frictional force that can be manually applied for a given task. The use of compressible elevated features also importantly reduces the level of force required by the user to securely grip the handle of the surgical instrument and achieve the required gripping force for a particular task. The repetitive awkward motions that surgeons employ for certain procedures can lead to excessive strain and injury as the arm and shoulder rotation needed for proper instrument position and movement over many hours can be limiting. Tissue manipulation during surgery generally requires a more sensitive application of force. The grasping or working of bone features and surfaces, for one example, can require one level of frictional engagement of the handle, the grasping of a blood vessel a much lighter frictional engagement, and the grasping of objects introduced into the surgical space can vary significantly depending on the surface characteristics and whether these surfaces are wet or dry.

For many applications, hand held surgical instruments can be simultaneously used to perform a procedure in which the primary hand of the surgeon manipulates a first instrument and the secondary hand of the surgeon manipulates a second instrument in a coordinated movement to perform required operative procedures. In one example, the surgeon will grasp a suturing needle in their primary hand, that may either move or remain stationary during all or a portion of the suturing procedure. In the case where suturing involves closing a wound in which a portion of skin at one side of the wound is to be sutured to skin on the opposite side of the wound, the skin must be grasped with sufficient force to overcome the inherent tension of the skin but without causing damage to the skin tissue. In one example, forceps can also be used with a surgical scalpel or scissors that are held in the primary hand to cut tissue held and/or positioned with the forceps.

A preferred embodiment provides an integral molded body that includes flexible arms that provide for rotation about one or more axes of rotation. The user can apply pressure with their fingers to compress the arms, and the attached tips of the forceps, towards each other to grasp tissue. The user can release pressure such that the spring force of the arms causes the arms to separate. The amount of resilience can be selected during manufacture depending upon the application.

This present invention further relates to a forceps that can be attached to a forceps. The arm adapter provides rounded arm elements that are easier to hold, thereby giving the surgeon greater control and ability to more precisely manipulate objects or tissues. The adapter of the present invention includes a cavity extending between the arms that receives the proximal end of the forceps such that the tips extend from the distal end of the adapter. An outer surface region of the adapter includes handle elements that are gripped manually by the hand of the user during a surgical procedure. The handle elements have contoured surfaces to provide for proper orientation of the tips of the forceps relative to the surgical site. The contoured surface of the handle elements can include a proximal section with a thicker diameter that tapers to a smaller diameter section at the distal end. The distal section can have a recess for the thumb of the user. The tips can be rigidly oriented relative to the surface features to improve manual manipulation. The handle elements can be shaped for left- or right-handed use. Preferred embodiments are fabricated in which the handle elements are wider that the metal arms so as to minimize the amount of metal, such as stainless steel, that is required yet still retain the desired handle geometry and size.

In accordance with a preferred embodiment, the method for using the molded body of the present invention involves selecting a forceps, connecting it or inserting it into the molded body, performing the selected procedure, removing the forceps for cleaning, sterilization, and reuse, and either sterilizing or disposing of the molded body.

In another preferred embodiment, the handle elements are grasping surface elements that are assembled with the forceps during manufacture. The elements in this embodiment can be made of a material suitable for sterilization after use. The elements can be molded as unitary bodies with selected portions of the surface having a hatched, braided or dimpled surface to provide frictional surface regions unlikely to slip when grasped by the user even when wet. The grasping surface elements can comprise a pair of elastomeric bodies each with a contoured upper surface as described herein and a flat lower surface that can have a slot or channel to receive the metal portions of the forceps.

The forceps handle can be manufactured using an injection molding or 3D printing process. A molded plastic material may be used and can be adapted for single use (disposable), or alternatively, manufactured using a material suitable for sterilization procedures for reuse. The forceps handle elements can be color-coded such that the color can indicate a particular size and/or type of forceps for the type of procedure.

Many applications require the forceps to have a spring force that is preferably within a range that aids the surgeon in the sensation of force feedback during tissue manipulation. It is preferred in certain embodiments to apply limited forces of compression, as well as limited torque during rotation of the forceps, to avoid damaging the tissue. It is further desirable to have a sufficient spring force operating to separate the arms of the forceps to release the tissue in a predictable way. The two arms of the forceps preferably are not displaced laterally during rotation thereof while grasping tissue, that is, the arms must have sufficient lateral rigidity that they remain aligned with the application of torques directed in opposite rotational directions on each arm.

The surgical forceps enable the grasping of different sizes of objects or tissue features. The increase in surface area on the contoured grasping surfaces, and the resulting increase in tactile feedback, improves the range of force that can be applied.

### DESCRIPTION OF THE DRAWINGS

FIG. 1 is a side view of a contoured forceps surface in accordance with a preferred embodiment of the present invention.

FIG. 2 illustrates a plurality of contoured forceps gripping surfaces of different sizes in accordance with a preferred embodiment of the present invention.

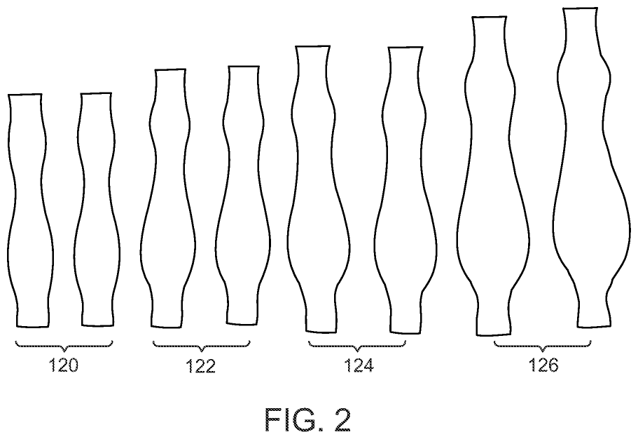

FIG. 3 illustrates side and front views of a contoured forceps gripping surfaces in the open position in accordance with a preferred embodiment of the present invention.

FIG. 4A shows side and front views of an embodiment of a contoured forceps gripping surfaces in the closed position, in accordance with a preferred embodiment of the present invention.

FIG. 4B shows side and front views of an alternative embodiment of a contoured forceps gripping surfaces in the closed position, in accordance with a preferred embodiment of the present invention.

FIG. 5A is a top view of a contoured forceps gripping surfaces having different sizes and the different size metal forceps on which they are mounted in accordance with a preferred embodiment of the present invention.

FIG. 5B shows different textured surfaces for contoured forceps gripping surfaces for preferred embodiments.

FIG. 6 is a side view of a contoured forceps having spaced apart small protrusion, trough and large protrusion textured regions to enhance grasping by the thumb and forefinger of the user in accordance with a preferred embodiment of the present invention.

FIG. 7 lists the grip lengths, feature widths and ratios of different size forceps gripping surfaces in accordance with a preferred embodiment of the present invention.

FIG. 8 illustrates side views of a contoured forceps with differing lengths and thicknesses of the gripping surfaces in accordance with a preferred embodiment of the present invention.

FIG. 9 includes side views of a contoured forceps in the open and closed positions, a top view and an enlarged side view of the region in which the arms of the forceps are joined, respectively, in accordance with a preferred embodiment of the present invention.

FIG. 10 is a perspective view from the top side of contoured forceps of different sizes and thicknesses in accordance with a preferred embodiment of the present invention.

FIG. 11 is a front side view of a contoured scalpel handle having a textured region to enhance grasping by the user in accordance with a preferred embodiment of the present invention.

FIG. 12 is a cross-sectional view of a contoured scalpel handle in accordance with a preferred embodiment of the present invention.

FIG. 13 is a view of a contoured dental instrument having a textured region to enhance grasping by the thumb and forefinger in accordance with a preferred embodiment of the present invention.

FIG. 14 is a cross-sectional view of a contoured dental instrument in accordance with some embodiments of the present invention.

FIGS. 15A and 15B illustrate top and side views, respectively, of a contoured forceps with ball end in accordance with some embodiments of the present invention.

FIG. 16A illustrates a contoured instrument handle having a textured region in accordance with some embodiments of the present invention.

FIG. 16B illustrates a magnified view of FIG. 16A.

FIG. 17A illustrates an alternative contoured instrument handle having a textured region in accordance with some embodiments of the present invention.

FIG. 17B illustrates a magnified view of FIG. 17A.

FIGS. 18A, 18B, and 18C illustrate alternative textured regions for use in accordance with some embodiments of the present invention.

FIGS. 19A and 19B illustrate a textured surface element before and after compression by a user's hand in accordance with some embodiments of the present disclosure.

FIGS. 20A and 20B illustrate an arrangement of textured surface elements on a contoured gripping surface in accordance with some embodiments of the present disclosure.

FIG. 21 illustrates a bottom view of a single arm of a contoured surgical forceps with the outline of a standard surgical forceps overlaid in accordance with some embodiments of the present disclosure.

FIG. 22 is a flowchart describing a method of manufacturing a surgical forceps in accordance with some embodiments taught herein.

### DETAILED DESCRIPTION OF THE INVENTION

The foregoing and other features and advantages of the systems and methods for using contoured forceps will be apparent from the following more particular description of preferred embodiments of the system and method as illustrated in the accompanying drawings in which like reference characters refer to the same parts throughout the different views.

A preferred embodiment of the invention relates to a forceps contoured gripping surfaces that can be attached to a formed metal forceps. The gripping surfaces can be a sterilizable or disposable, and include contoured plastic handle elements that can be used on existing forceps or attached to a formed metal forceps base. In addition to making the textured contoured forceps easier to hold, the textured compressible surfaces also have the advantage of making the handle less slippery than the traditional steel handle. The textured compressible forceps gripping surfaces are easier to manipulate thereby allowing the precision of the grasping and manipulation to be increased and to reduce user fatigue over the course of use. The textured surface features preferably have low porosity, particularly for sterilizable devices to provide for more durable use. Thus, the outer surface provides a fluid impermeable surface that is biocompatible.

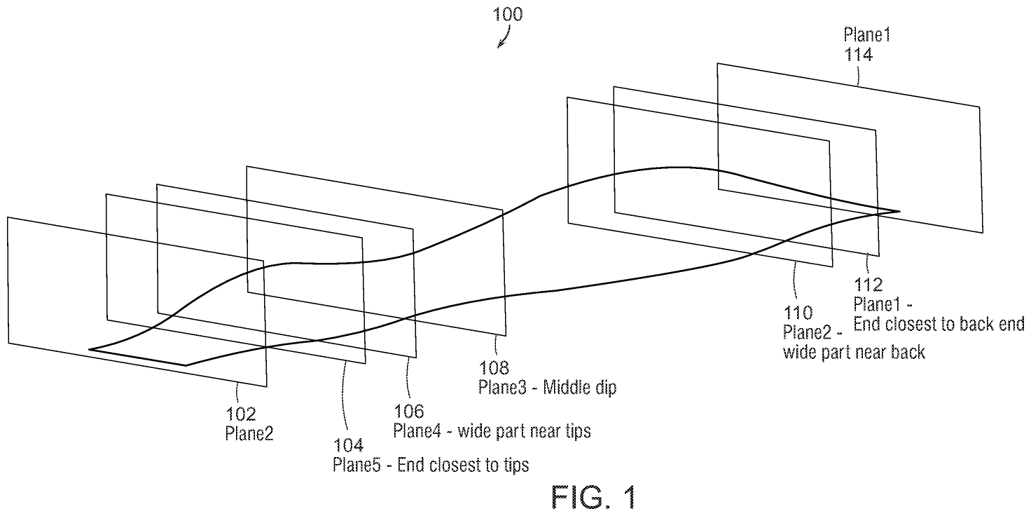

FIG. 1 illustrates a preferred embodiment of the invention where a handheld surgical instrument body having a contoured gripping surface 100 can be attached to a metal base or fabricated as a unitary body. FIG. 1 is a perspective view of one gripping surface, which is divided into a proximal portion with a first plane 114 passing across a proximal end, a distal portion with plane 102 at the distal end, a plane 104 across the distal inclined surface, and a plane 106 across the widest portion of the small protrusion. The central portion includes a plane 108 across the trough at the smaller diameter region where at least the thumb and forefinger of the user typically grasps the instrument. The large and small protrusion portions of the gripping surface have a generally circular or elliptical cross-sectional shape. Plane 110 extends across the widest portion of the gripping surface and plane 112 extends across the proximal sloping surface at the proximal end.

Between the distal and proximal wider portions is a concave section or trough adapted to be grasped by the thumb/forefinger and optionally also middle finger of the user. Between the widest portion and the proximal end is a second concave section or trough adapted to rest in the web of the user's hand between the thumb and forefinger.

The dimensions of the handle portion are selected to provide for precision and control of the tool, such as a blade, during use as well as comfort to the user. The dimensions of the handle features are also of importance as they maximize contact surface area to improve tactile feeling of the handle in the user's hand, which increases the user's sensitivity to the amount of gripping force being applied to the instrument such as a forceps. Two common grips of the forceps include squeezing between the thumb and forefinger and the pencil grip where the thumb, forefinger and middle finger of the user's hand provide three points of contact in the first trough section at plane 108. In a preferred embodiment, the first trough of each arm gripping surface has a partially circular or elliptical cross-section with a lateral diameter or width in the range of 8-18 mm depending upon the length, preferably in the range of about 9-15 mm. In a distal direction from the trough, the diameter of the handle increases to prevent the fingers from sliding distally toward the tips of the forceps extending distally, particularly when more force is exerted in a distal direction. The larger diameter distal portion at plane 106 has a flared diameter in the range of 10-18 mm, preferably about 12-18 mm. The largest diameter portion at plane 110 has a diameter in the range of 12.5-28 mm, preferably in a range of about 14-24 mm. The larger central portion increases the area of contact with the fingers to further stabilize the device in the user's hand. The proximal portion can be flatter in cross-sectional profile and can have a reduced diameter to taper onto a metal portion of the device or have a diameter in the range of 8-14 mm.

The textured grasping material preferably will compress between about 1-20% of its thickness upon being grasped by the user, with the level of compression increasing with a firmer grip. Generally elastomeric polymers or silicones can be used which compress within this range at room temperatures. Generally, elastomeric materials that have a compression set (ASTM standard) in a range of 1-20% over the temperature range of 0-100 degrees centigrade that are also biocompatible and fluid resistant can be patterned on the grip surface with a coefficient of friction suitable for the surgical instruments described herein.

Preferred embodiments described herein utilize surface elements that compress in a range of 4-15% of the height above a base level of the handle surface. In further preferred embodiments, this range is between 6-12% of this height. This level of compressibility enables the user to firmly grasp the handle surface over a variable range of applied force. The raised elements can have a dome shaped contour, for example, to have a small region of maximum elevation that deforms to expand in size to increase the load-bearing surface that also increases the amount of friction relative to the gloved fingers of the user's hand. This deforming characteristic further increases the surface contact area, which the user can sense with increased pressure.

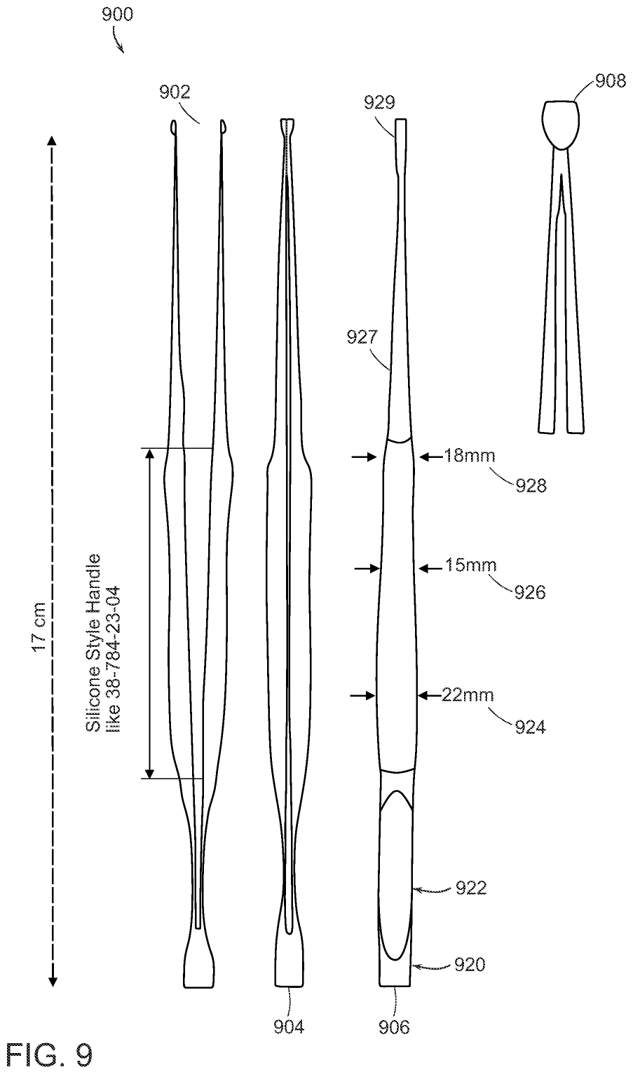

The dimensions are correlated with the longitudinal distance between features to achieve the described fit to the user's hands. The grip length can be in the range of 48-66 mm. The ratio of large protrusion width to length of the gripping surface is in the range of 0.24 to 0.26 and is preferably about 0.2545 for the different length forceps as illustrated in FIG. 7.

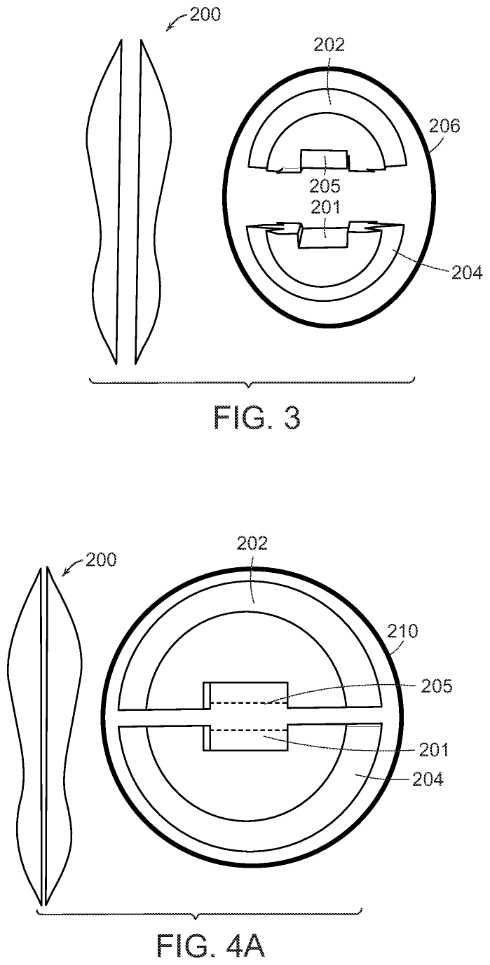

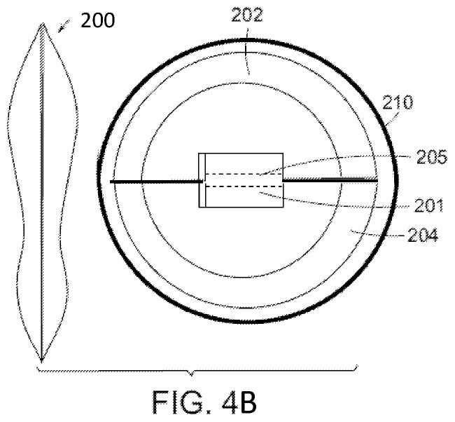

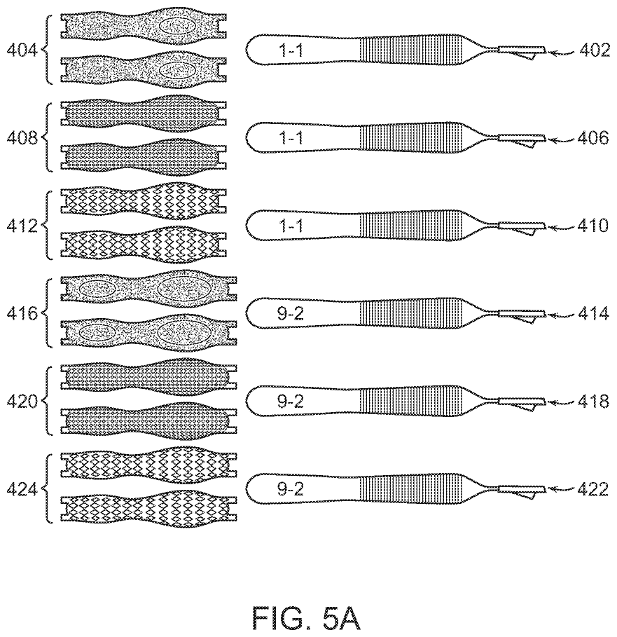

An important factor in surgical applications is the need to rotate the forceps, frequently with the secondary or second hand of the surgeon. As seen in FIG. 3, the side view of a forceps gripping surfaces 200 corresponds to the arms of the forceps in the separated position with arm 202 being separated from arm 204 to fall within an elliptically shaped 206 gripping position. Note in this embodiment the gripping surfaces are attached to metal arms 205 and 201, respectively, where portions of the metal arms are within slots or channels in the lower surface of the elastomeric body. The gripping surface elements can be attached to the metal arms by a snap attachment that enables later separation, or by an adhesive. As one arm rotates through a 20-60 degree angle, for example, the second arm generally rotates through the same angle but can experience an oppositely directed force imparted by the grasped object to both arms, which may be connected to the body of the patient. FIG. 4A shows the two arms of the forceps 200 in the closed position with the tips in contact. The grasping of skin, a ligament, an artery, or a bone can result in such forces being imparted to the forceps. FIG. 4B shows an alternative embodiment of the forceps 200 where the gripping surfaces 202, 204 attached to different arms come into contact with one another when the tips of the forceps 200 are closed. When the gripping surfaces 202, 204 come into contact, a substantially circular profile is obtained in the end view. In some embodiments, the contact between gripping surfaces produces an unbroken circular perimeter. By producing a circular profile, the instrument is easy to roll back and forth or rotate about a longitudinal axis in the user's hand while gripping force is maintained.

Precise control of the forceps during use is critical. If the handle is awkward to hold, this can contribute to fatigue and the difficulty of the procedure by compelling the user to awkwardly grasp the tool to achieve the proper angle and level of force required to grasp and manipulate the object or tissue as needed. Thus, proper balance, good contact between the user's hand and the handle, and the ability to rotate the handle without having to grip the tool differently can be of great importance. Further, in contrast to traditional flat sided forceps, which can only be rotated approximately 60 degrees without moving hand position, the rounded contours of the gripping surfaces allow for the forceps to be comfortably rotated up to 90 degrees.

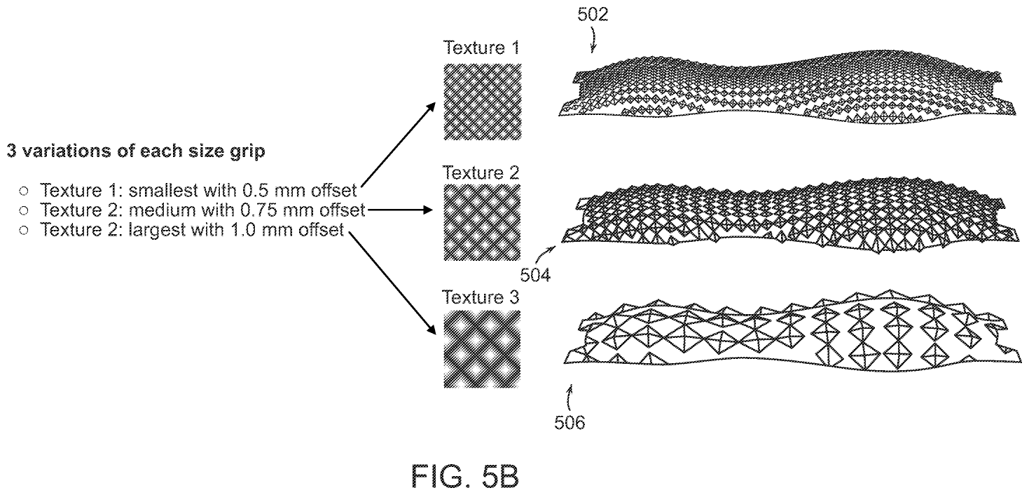

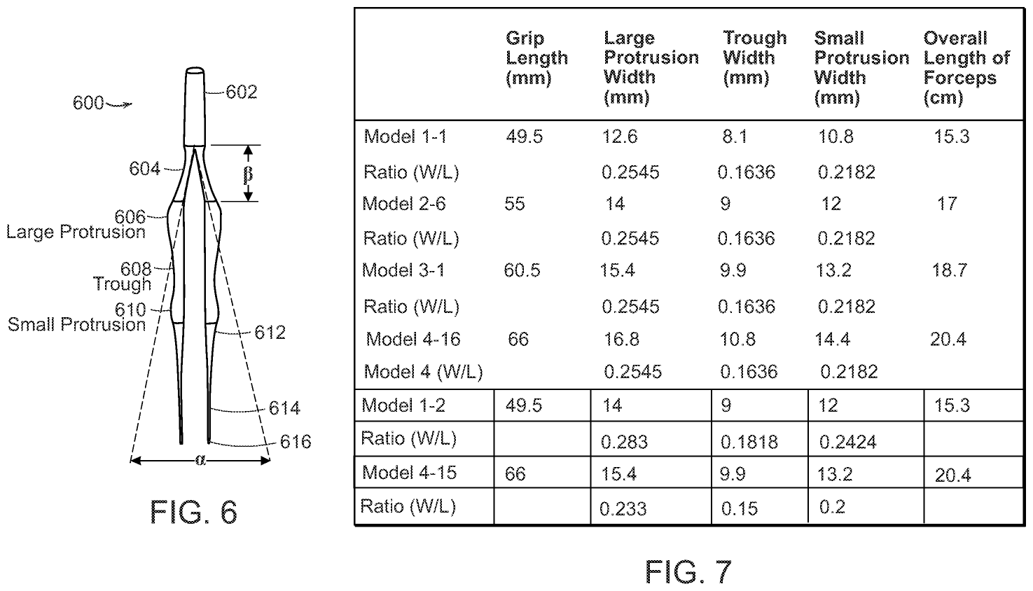

FIG. 5A illustrates a plurality of different size grasping surface elements 404, 408, 412, 416, 420, and 424 that are fabricated in pairs to conform to existing sizes of commercial forceps 402, 406, 410, 414, 418 and 422 respectively. The grasping surface elements 404-424 can have smaller enlarged sections (plane 106) that are positioned closer to the tips of the respective forceps, and the largest diameter portions (plane 202) are situated over the smaller diameter portions of the commercial forceps. The textured surface elements can snap onto the commercial forceps with prongs that extend distally and proximally from the textured surface elements, and/or can also be attached with an adhesive. As seen in FIG. 5B, there are a plurality of different textures that can be used for different gripping surface elements. Texture 502 has about a 0.5 mm offset between raised features that compress under pressure applied by the hands and/or fingers of the user. Texture 504 has about a 0.75 mm offset between raised elements or nodules and texture 506 has about a 1.0 mm offset spacing between adjacent raised surface elements. Thus, the spacing between elements can vary from a 0.2 mm offset to about a 1.5 mm offset. The spacing and elevation of the raised elements can vary over different portions of the grasping surface to improve the haptic indication to the user of how much force is being applied, either by compression or lateral movement of the elevated portions of the surface elements in combination with compression.

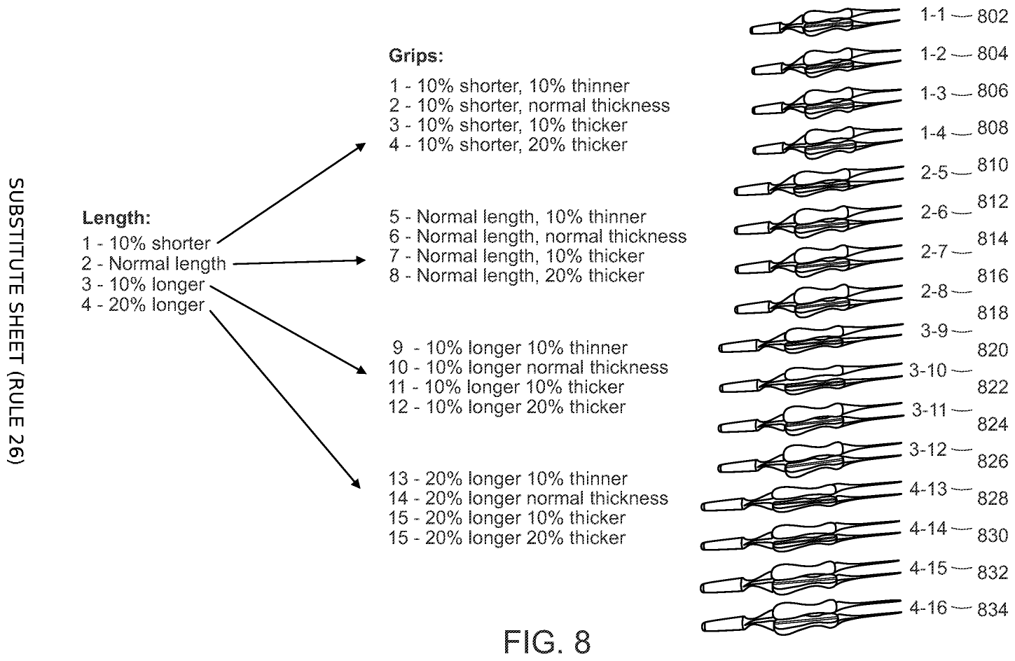

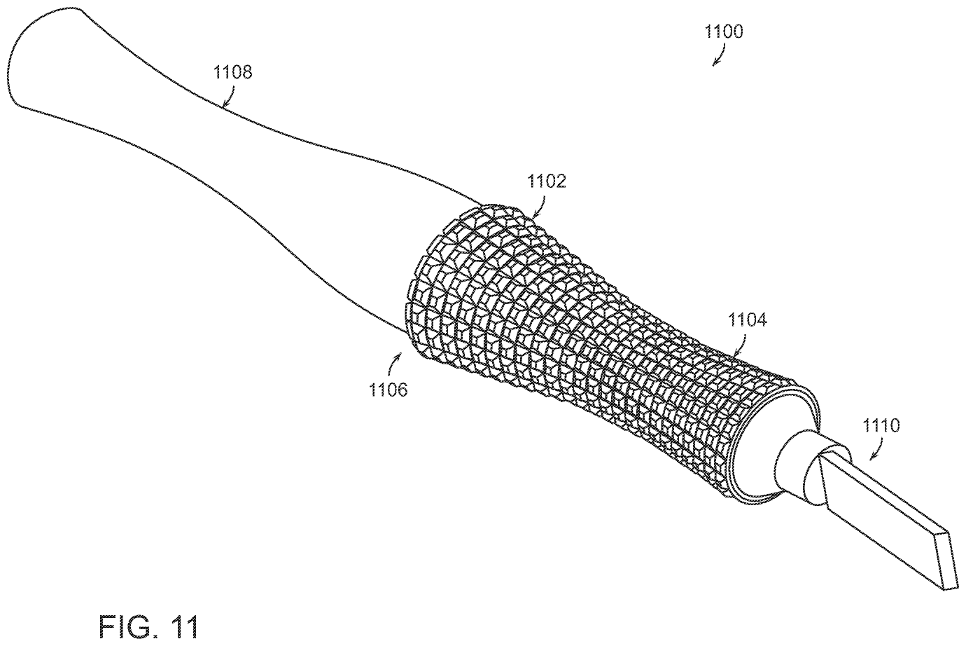

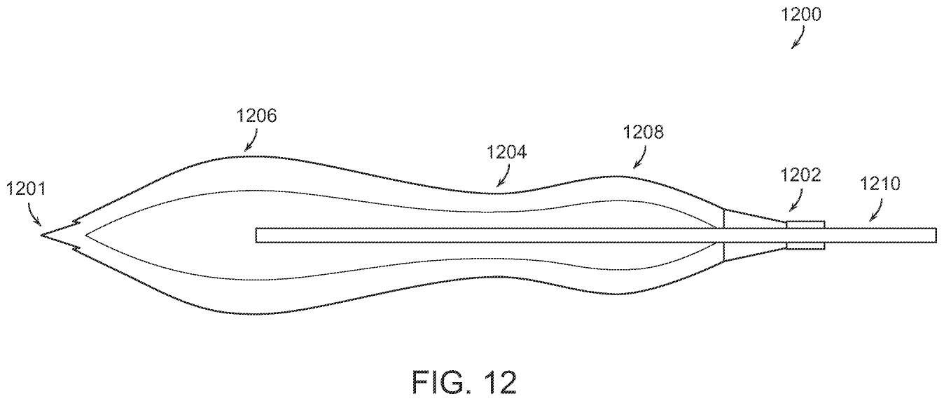

In reviewing the different proportions of forceps, a number of variations of design parameters were fabricated and tested for balance, weight and size to identify preferred configurations. FIG. 10 illustrates a plurality of different lengths and thicknesses of the grasping surface region that were fabricated by 3D printing using a nylon material and evaluated for preferred characteristics. An exemplary embodiment is depicted in FIG. 6. This embodiment had a proximal region 602 where the arms were joined at the proximal end to accommodate the desired flex of the arms between open and closed positions at the tip 616. The arms separate from region 602 by a distance ß at a joint angle «, wherein this angle can be greater than 10 degrees, for example, between 10-40 degrees. The length of the gripping surface extends from plane 112 and the largest protrusion 606 through trough 608 to the small protrusion and onto the forward slope at plane 104. The forward slope reduced in diameter at 612 to extend along rigid section 614 to the tip at 616. Note that the tips can optionally be fabricated with outwardly extending fixtures that can be used to grasp and manipulate sternum plates or other objects positioned within the surgical field (see the tips, for example, at 902 in FIG. 9). In one embodiment, such fixtures can operate the fixtures from the tissue clamp described in U.S. Pat. No. 7,901,420, the entire contents of which is incorporated herein by reference. The longer the forceps, the grip surface length was increased proportionately. The ratio of width of the gripping surface at the two larger diameter widths and at the trough to the gripping surface length generally fell within preferred ranges of 0.24-0.26 for the large protrusion (preferably about 0.2545), between 0.2 and 0.22 for the small protrusion (preferably about 0.2182) and between 0.15 and 0.18 at the trough (preferably about 0.1636).

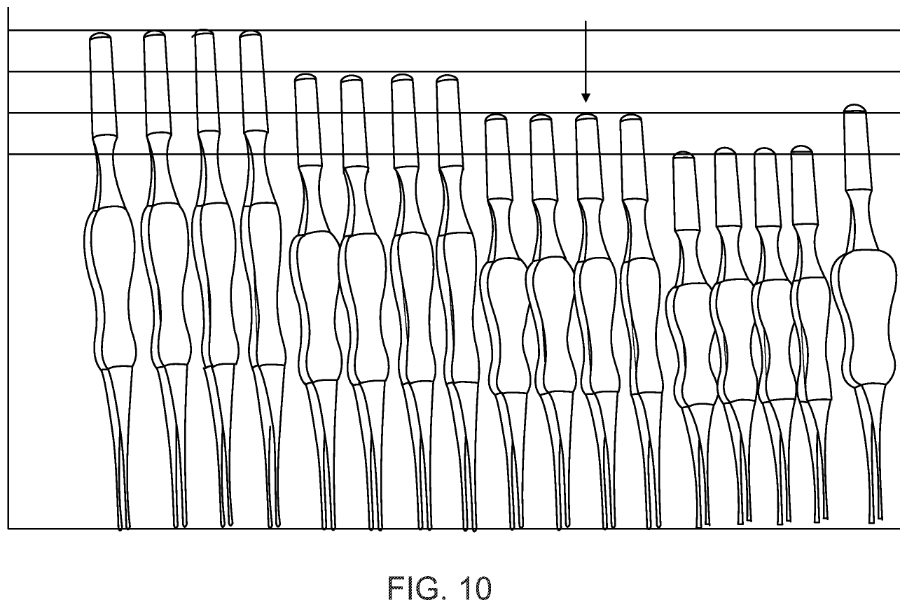

Shown in FIG. 8 are the preferred sizes of forceps in which different lengths have different associated thicknesses of the gripping surface region. Thus, a first length has sizes 802-808, the normal length has thicknesses 810-816. The next size being 10% longer than normal has thicknesses 820-826 and the 20% longer embodiment has thicknesses 828-834.

FIG. 9 illustrates a further preferred embodiment 900 including a roughened surface region to improve the frictional grasp of the forceps. In this embodiment the tips 902 of the forceps 922 are shown open, and also closed in the side view at 904. The grasping surface can comprise an elastomeric silicone with a smaller protrusion 928 (e.g., 18 mm), a narrower trough 926 (e.g., 15 mm) and a larger diameter protrusion 924 (e.g., 22 mm). The arms 927 are joined at proximal end 906 in the top view. The handles of the various embodiments of the present invention can include roughened surface region to enhance the grasp of the user. The forceps can include an elastic material that can be replaced when worn away or if a different shape, color or texture is needed for a particular procedure. The elastic material can slide onto the arms, for example, which can be made much smaller in certain embodiments to reduce the amount of metal needed to stiffen a composite metal forceps using the compressible elastomeric gripping surface described generally herein.

This embodiment can utilize the contoured shapes used in other embodiments, or alternatively, can utilize the shape having a proximal trough, a wider central region, and a smaller distal region. The elements can be bonded to the outer surfaces of the tongs or arms of a forceps. The user can then grasp and compress the arms from an open position to a closed or grasping position. The spacing between the arms will be reduced when grasping an object, but are generally not abutting when in use. The grasping metal elements can also comprise separate plastic elements in another embodiment.

The plastic contoured elements are attached by adhesive or snap-on elements to the metal arms in this embodiment and do not substantially alter the spring characteristic thereof. The cavity that separates a first arm from a second arm extends through the distal portion, the central portion, and into the proximal portion. In certain embodiments, the tips may be integrated into the handle using an adhesive, a snap-fit, or by mounting with hardware such as a screw. The tips may be inset into the handle at ridges.

In accordance with certain embodiments, the textured region can comprise larger diameter raised nubs or protrusions of greater thickness. As the user rotates the handle, the unbalanced force imparted by the user's hand to an edge of a nub causes that edge and the portion of the nub near the edge to compress. In this way, the nubs can provide feedback to the user's hand about the rotational attitude of the forceps as it is being held and moved by the user. This palpable feedback increases the user's ability to make fine-tuned adjustments to the rotation of the forceps while grasping and manipulating objects.

The method of grasping a surgical forceps having a first contoured surface and a second contoured surface may be performed, for example but not limited to, grasping a surgical forceps having a roughened surface or a textured surface. In accordance with certain embodiments, the grasping step may be performed with a user's secondary hand (i.e., their non-preferred hand or “off” hand). In accordance with certain embodiments, the method may include an additional step of performing a surgical procedure using a tool in the primary (i.e. dominant) hand of the user while simultaneously manipulating tissue or objects with the surgical forceps.

Methods for assembling a surgical forceps are further described in U.S. Pat. No. 10,201,362, the entire contents of which is incorporated herein by reference. The method can include fabricating a forceps handle having a first arm and a second arm, the first arm having a first contoured surface and the second arm having a second contoured surface such that the forceps handle can be held in a hand of a user, each arm having a contoured surface that includes a distal trough, a larger central portion and a proximal trough extending along a longitudinal axis of each arm. The step of fabricating a forceps handle having a first arm and a second arm, the first arm having a first contoured surface and the second arm having a second contoured surface such that the forceps handle can be held in a hand of a user, each arm having a contoured surface that includes a distal trough, a larger central portion and a proximal trough extending along a longitudinal axis of each arm may be performed, for example but not limited to, using an injection molding process for metal of the elastomeric components or a 3D printing process for such components. In accordance with various embodiments, the first arm and the second arm of the fabricated handle may comprise compressible textured or roughened surfaces at their proximal ends. These textured or roughened surfaces may comprise raised nobs, hexagonal protrusions, or diamond-shaped protrusions.

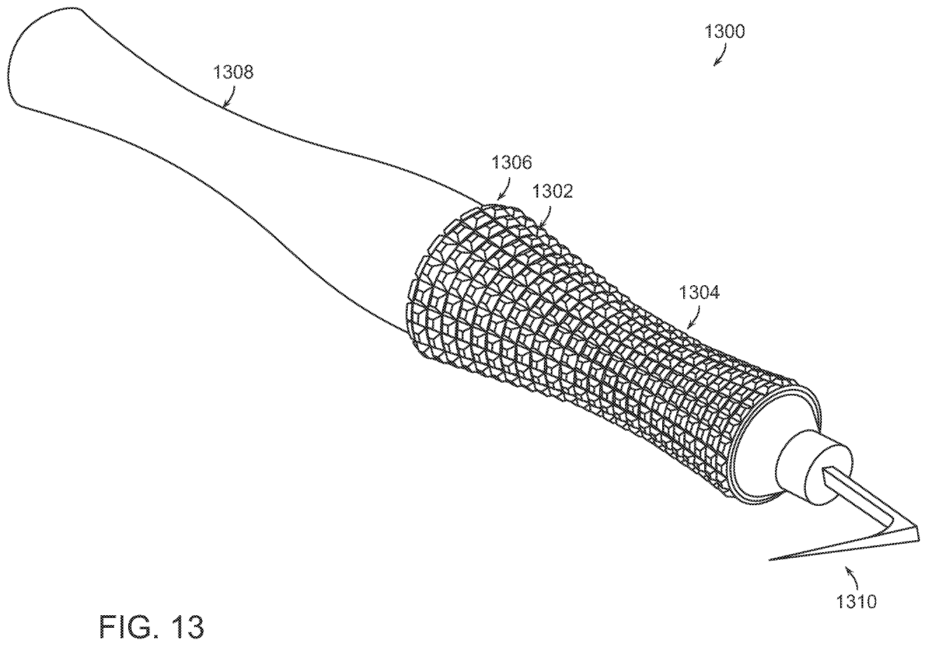

FIG. 11 illustrates a front side view of an ergonomic, contoured scalpel handle 1100 in accordance with various embodiments described herein. The scalpel handle 1100 can include one or more protrusions 1106 and one or more troughs 1104, 1108. The trough or troughs 1104, 1108 can be adjacent to the protrusion or protrusions 1106 in a longitudinal direction down the handle. The scalpel handle can include a post 1110 extending from a distal end of the scalpel handle 1100. The post 1110 is suitable to receive a scalpel blade. For example, the post 1110 can include fixation elements such as snap-on features or screws that match with complementary features on the scalpel blade.

The scalpel handle 1100 can include a contoured gripping surface 1102 that extends longitudinally along at least a portion of the scalpel handle 1100. The gripping surface 1102 can include texturing as described above in relation to at least FIG. 5B.

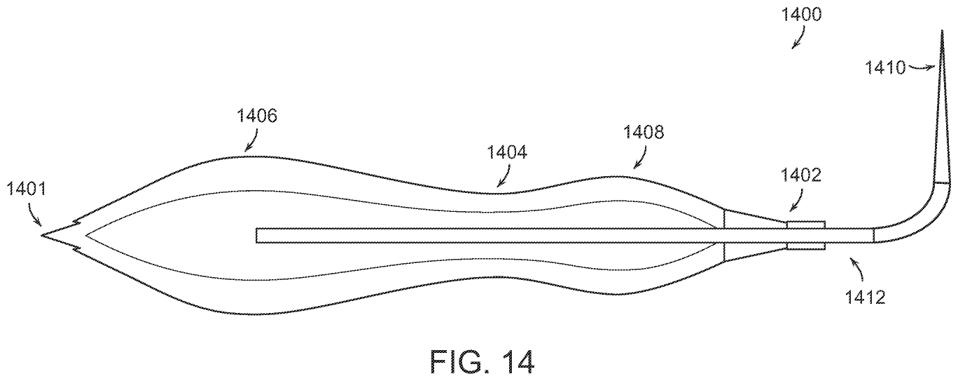

FIG. 12 illustrates a cross-sectional view of an ergonomic, contoured scalpel handle 1200 in accordance with various embodiments described herein. The scalpel handle 1200 can include one or more protrusions 1206, 1208 and one or more troughs 1204. In some embodiments, the scalpel handle 1200 can include (in order longitudinally from a proximal end 1201 to distal end 1202) a larger protrusion 1206, a trough 1204, and a smaller protrusion 1208. The scalpel handle 1200 can include a post 1210 that extends distally to the distal end 1202 of the handle 1200. The post 1210 is suitable to receive a scalpel blade. The post 1210 also extends into the interior of the handle 1200. In some embodiments, the post 1210 extends into the handle 1200 by more than half or, more preferably, more than two-thirds of the distance between the distal end 1202 of the device 1200 and the proximal end 1201 of the device 1200.

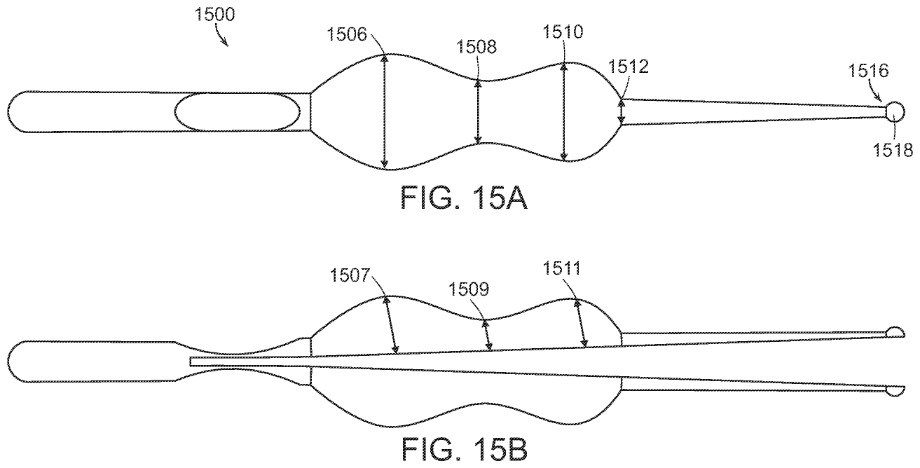

FIG. 13 illustrates an ergonomic, contoured dental instrument 1300 in accordance with various embodiments described herein. Although the dental instrument 1300 depicted in FIG. 13 is a file, one of ordinary skill in the art would understand that other dental instruments gripped by the hand would similarly benefit from the handle designs disclosed herein with respect to the file. Such other dental instruments can include, but are not limited to, scrapers, drills, mirrors, sickle probes, pliers, tongs, spoons, water dispensers, air dispensers, or scalpels. The dental instrument 1300 can include one or more protrusions 1306 and one or more troughs 1304, 1308. The trough or troughs 1304, 1308 can be adjacent to the protrusion or protrusions 1306 in a longitudinal direction down the instrument. The dental instrument can include a file 1301 or rasp extending from a distal end of the dental instrument 1300.

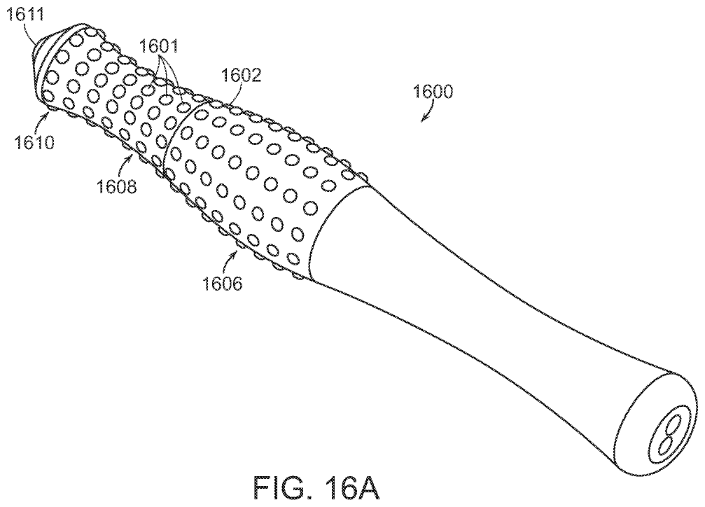

FIG. 14 illustrates a cross-sectional view of a dental instrument 1400 in accordance with various embodiments described herein. The dental instrument 1400 can include one or more protrusions 1406, 1408 and one or more troughs 1404. In some embodiments, the dental instrument 1400 can include (in order longitudinally from a proximal end 1401 to distal end 1402) a larger protrusion 1406, a trough 1404, and a smaller protrusion 1408. The dental instrument 1400 can include a file 1410 attached to a post 1412 that extends distally to the distal end 1402 of the instrument 1400. The post 1412 also extends into the interior of the instrument 1400. In some embodiments, the post 1412 extends into the instrument 1400 by more than half or, more preferably, more than two-thirds of the distance between the distal end 1402 of the instrument 1400 and the proximal end 1401 of the instrument 1400. In some embodiments, the file 1410 is separable from the post 1412. For example, the file 1410 can engage with the post 1412 by screw threads. The file 1410 can be removed for sterilization and/or replacement with a new file tip. In some embodiments, the dental instrument 1400 can have multiple interchangeable instrument ends of which the file is only one.

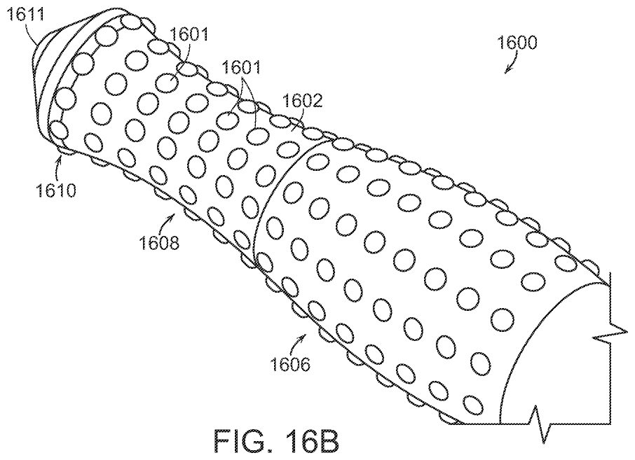

FIGS. 15A and 15B illustrate top and side views, respectively, of a contoured forceps 1500 with ball end in accordance with some embodiments of the present invention. Each arm of the forceps 1500 includes a contoured gripping surface (i.e., a first arm includes a first contoured gripping surface and a second arm includes a second contoured gripping surface). When the arms are brought together by application of a pinching motion, the first and second contoured gripping surfaces come together to produce a substantially elliptical or circular profile when viewed end on. Each contoured gripping surface includes a proximally located protrusion or bulge with a first width 1506, a narrower trough having a second width 1508, and a distal protrusion or bulge having a third width 1510. The third width 1510 is smaller than the first width 1506. The first width 1506 and the third width 1510 are greater than the second width 1508. The first width 1506, the second width 1508, and the third width 1510 are all greater than a width 1512 of the arms of the forceps 1500 at a distal end of the contoured gripping surface in some embodiments. Each protrusion and trough of the contoured gripping surface can also be described by a thickness as illustrated best in the side view of FIG. 15B. A first thickness 1507 of the proximal protrusion is larger than a third thickness 1511 of the distal protrusion. A second thickness 1509 of the trough is less than the first thickness 1507 and the third thickness 1511 at the protrusions. In forceps embodiments where the contoured gripping surfaces produce a circular profile when the arms are brought together, the thickness of each protrusion or trough is about half of the respective width. The forceps 1500 includes ball end tips 1518 that are adjacent to a neck 1516 portion of the forceps 1500.

In some embodiments, the first width 1506 of the proximal protrusion can be in a range from 16 to 22 mm. In some embodiments, the second width 1508 of the central trough can be in a range from 10 mm to 15 mm. In some embodiments, the third width 1510 of the distal protrusion can be in a range from 13 mm to 18 mm. In some embodiments, the width of the neck portion 1516 can be about 2 mm. In some embodiments, a width of the balls at the ball tips can be about 4 mm. In some embodiments, the width 1512 of the arms at the distal end of the contoured gripping surface can be about 5.5 mm.

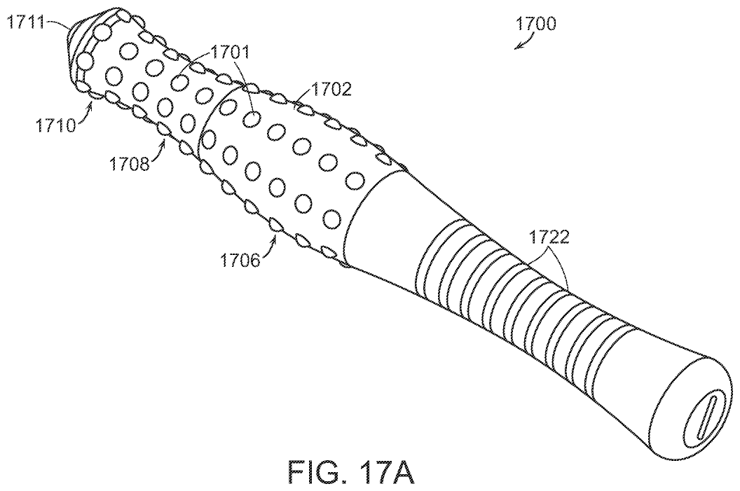

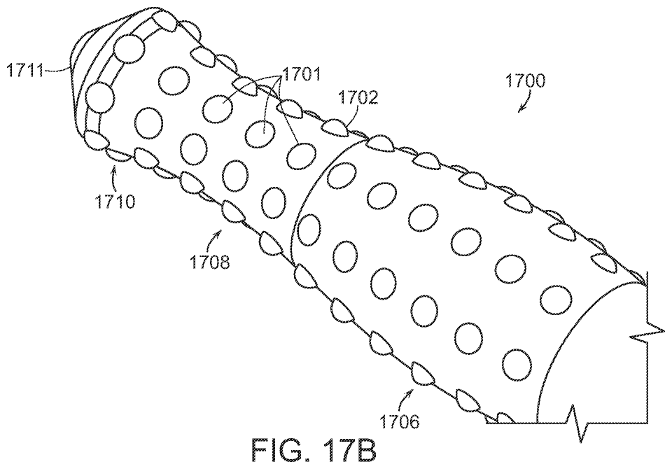

FIG. 16A illustrates a contoured instrument handle 1600 having a textured region in accordance with some embodiments of the present invention. The handle 1600 includes a proximal protrusion or bulge 1606, a trough 1608, and a distal protrusion or bulge 1610. A distal end 1611 can include a post (as described above such as with respect to FIG. 11) or other mounting device to enable connection of a blade or tool. For example, the distal end 1611 can include a threaded receptacle to connect with a complementary screw end of the tool. The handle 1600 includes a contoured gripping surface 1601 that extends over at least a portion of the surface of the handle 1600. In some embodiments, the contoured gripping surface 1602 can include textured surface elements 1601. FIG. 16B illustrates a magnified view of FIG. 16A that shows the textured surface elements 1601 in greater detail. In some embodiments, the textured surface elements 1601 can include raised bumps, nubs, nodules or small protrusions. In some embodiments, the contoured gripping surface 1602 can be applied to the handle 1600 by using an overmolding technique. As shown in FIG. 16B, the individual surface elements 1601 can include a dome-like structure with a flat plateau top surface. In some embodiments, a raised ridge or ring may surround the base of each individual textured surface element 1601.

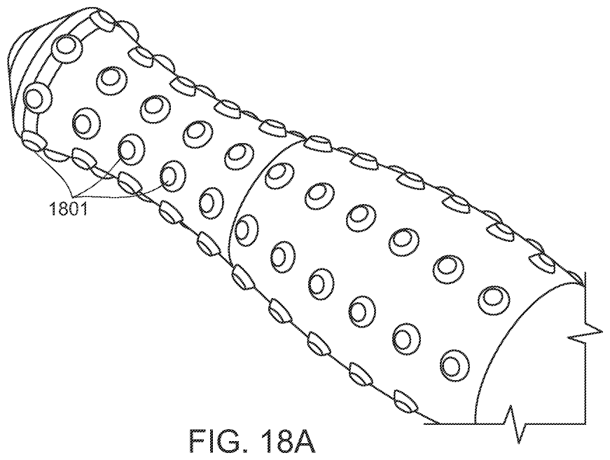

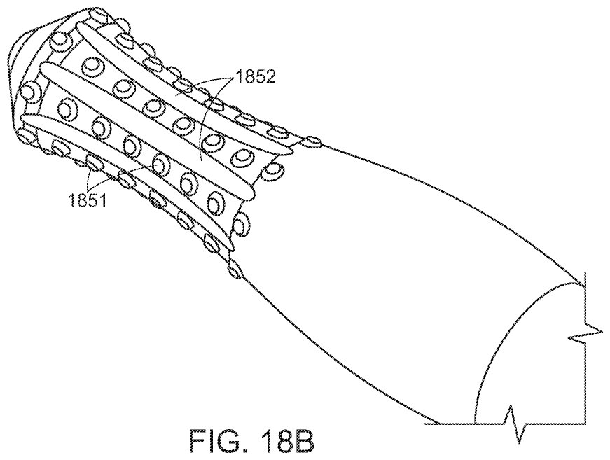

FIG. 17A illustrates an alternative contoured instrument handle 1700 having a textured region in accordance with some embodiments of the present invention. FIG. 17B illustrates a magnified view of FIG. 17A. The handle 1700 includes a proximal protrusion or bulge 1706, a trough 1708, and a distal protrusion or bulge 1710. A distal end 1711 can include a post (as described above such as with respect to FIG. 11) or other mounting device to enable connection of a blade or tool. The handle 1700 includes a contoured gripping surface 1702 that extends over at least a portion of the surface of the handle 1700. In some embodiments, the contoured gripping surface 1702 can include textured surface elements 1701. The handle 1700 can include ridges 1722 located proximally with respect to the proximal protrusion 1706. The ridges 1722 can extend outward from a surface of the handle 1700 or can extend inward from the surface of the handle 1700. For example, the ridges 1722 can be formed during molding of the handle or can be cut into the handle.

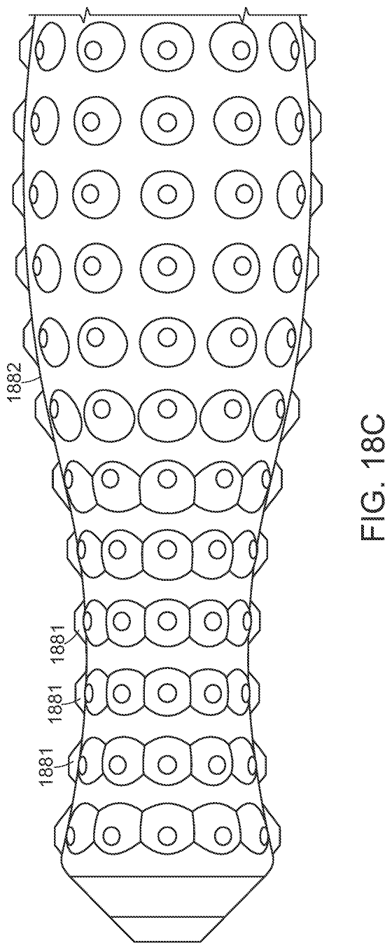

As compared to the textured surface elements 1601 of handle 1600, the textured surface elements 1701 of handle 1700 are larger in size. The textured surface elements 1701 are dome-shaped with a rounded profile and no flat top. FIGS. 18A, 18B, and 18C show other alternative arrangements of textured surface elements 1801, 1851, 1881 that can be used in combination with, or in place of, the textured surface elements 1601, 1701 in handles or forceps of this disclosure. The textured surface elements 1801 are similar in size to the elements 1701 shown in FIG. 17B but include the flat plateau top surface of the elements 1601 shown in FIG. 16B. The textured surface elements 1851 are organized in longitudinal columns that are interspaced around the perimeter of the contoured gripping surface by sections that do not have textured surface elements 1851. In some embodiments, the interspaced sections can include depressions 1852 or ridges. As shown in FIG. 18B, the textured surface elements 1851 cover only a portion of the contoured gripping surface, namely, a portion of the distal protrusion and the trough. The textured surface elements 1851 and depressions 1852 do not cover the proximal protrusion. In FIG. 18C, the textured surface elements 1882 have varying inter-element spacing. In some embodiments, neighboring textured surface elements 1882 can abut or conjoin with one another as the inter-element spacing becomes small. In some embodiments, the textured surface elements 1882 elements are arranged into columns (i.e., along the longitudinal axis) and into rows or rings (i.e., around the circumference of the instrument). In particular, the inter-element spacing can be lower in portions of the handle where the characteristic dimension (e.g., diameter) is smaller, such as the trough, while the inter-element spacing can be greater in portions of the handle where the characteristic dimension is larger such as the proximal bulge or protrusion and distal bulge or protrusion.

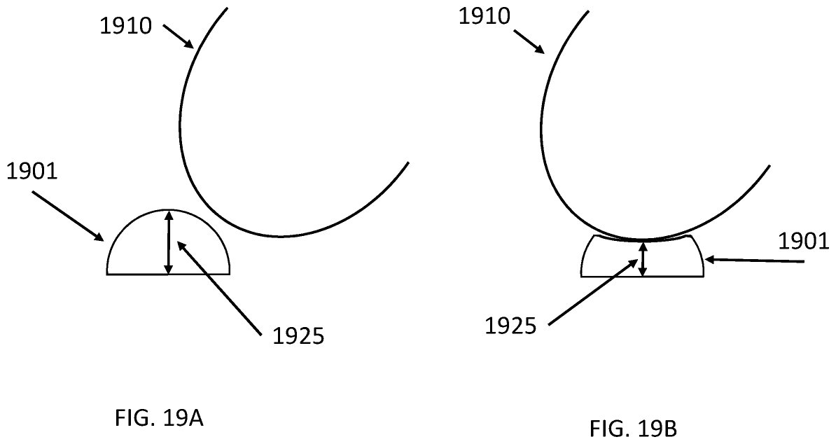

FIGS. 19A and 19B illustrate a textured surface element 1901 before and after compression by a user's hand 1910 in accordance with some embodiments of the present disclosure. The textured surface element 1901 is illustrated as a dome-shaped raised element, but it should be understood that other shapes are possible in view of the disclosure above. In FIG. 19A, the hand 1910 (which could be a finger as shown or a different portion of the hand such as the palm) has not contacted the textured surface element 1901. Thus, the textured surface element 1901 is still in an original, uncompressed state with a given uncompressed characteristic dimension 1925, such as height, radius, length, or width. In FIG. 19B, the hand 1910 has gripped the instrument and contacted the textured surface element 1901. The textured surface element 1901 is now compressed and has a compressed height 1925. In some embodiments, the height 1925 of the textured surface element 1901 can be compressed to a value in a range of 10-20% less than the uncompressed height 1925. In some embodiments, the compressed height 1925 can be in a range of 4-15%, in a range of 6-12%, or about 8% less than the uncompressed height. In some embodiments, a circumference of an uncompressed textured surface element 1901 can be about 0.710 inches (18.03 mm) while the circumference of the compressed textured surface element 1901 can be about 0.658 inches (16.71 mm).

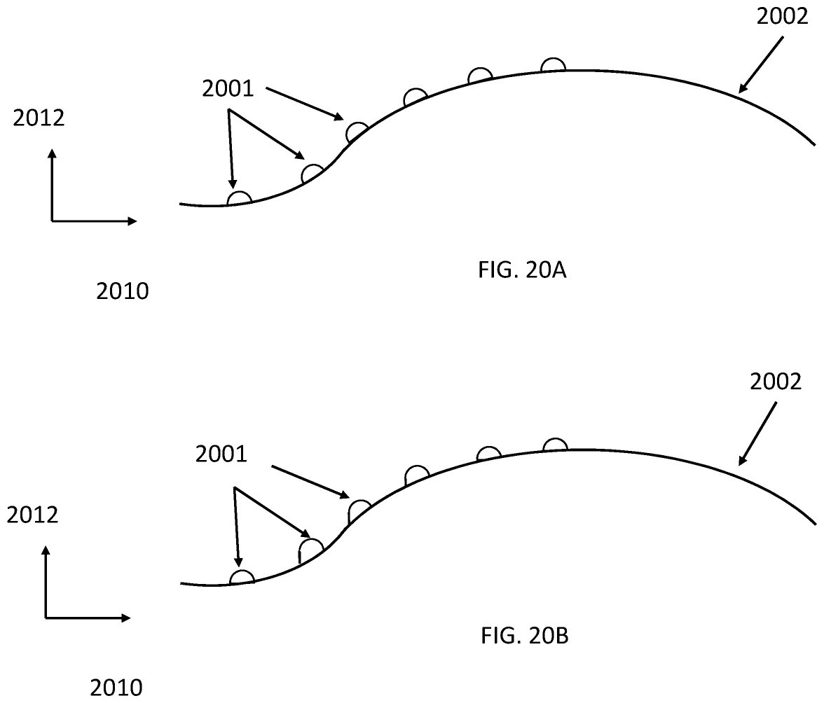

FIGS. 20A and 20B illustrate cross-sectional views of alternative arrangements of textured surface elements 2001 on a contoured compressible grasping surface 2002. In FIG. 20A, the textured surface elements 2001 are aligned with the local curvature of the contoured surface 2002. In other words, each textured surface element 2001 extends in a direction that is normal from the surface 2002 at the location of the surface element 2001 without regard for a longitudinal axis 2010 or radial axis 2012 of the instrument. In some embodiments, each textured surface element 2001 extends a same dimension (e.g., height) from the surface 2002. FIG. 20B illustrates an alternative arrangement wherein the texture surface elements are aligned with a radial axis 2012 of the instrument. This also creates directional alignment between neighboring textured surface elements 2001. The aligned textured surface elements 2001 may have different dimensions (e.g., heights) with respect to one another. A given textured surface element 2001 that is aligned with an axis such as the radial axis 2012 can have a varying height from the proximal portion of the element 2001 to the distal portion of the element 2001.

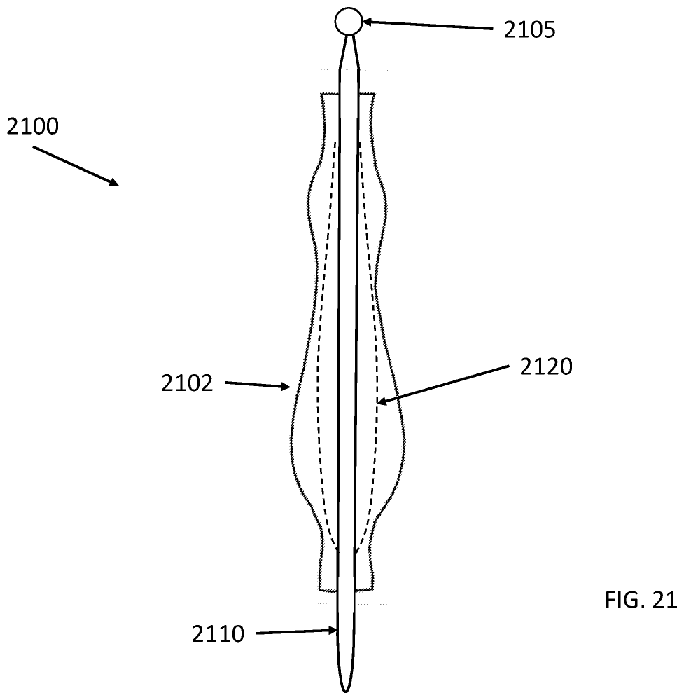

FIG. 21 illustrates a bottom view of a single arm 2100 of a contoured surgical forceps in accordance with some embodiments described herein. The arm 2100 includes a support arm 2110 with a ball tip 2105 at a distal end. The support arm 2110 is embedded within or attached to a contoured grasping surface 2102 as described in detail in embodiments above. The support arm 2110 may be made of a relatively more rigid material such as metal including stainless steel. The support arm 2110 can have a substantially constant or even tapered (not shown) width in the portion of the support arm 2110 that passes through the contoured grasping surface 2102. The width of the support arm 2110 is smaller than the width of the contoured grasping surface 2102. This shape and arrangement contrasts with a conventional forceps such as an Adson forceps shown in outline 2120 overlaid on the arm 2100. The conventional forceps includes widened or broadened arms to provide sufficient surface area for the user to manipulate the conventional forceps. However, the increased amount of material (usually metal) needed to produce these broadened sections can lead to additional weight and materials expense that may be undesirable. By removing the broadened sections and having the user grip the contoured grasping surface, the resulting instrument can be more inexpensive to produce and may be lighter weight, which can contribute to reducing fatigue.

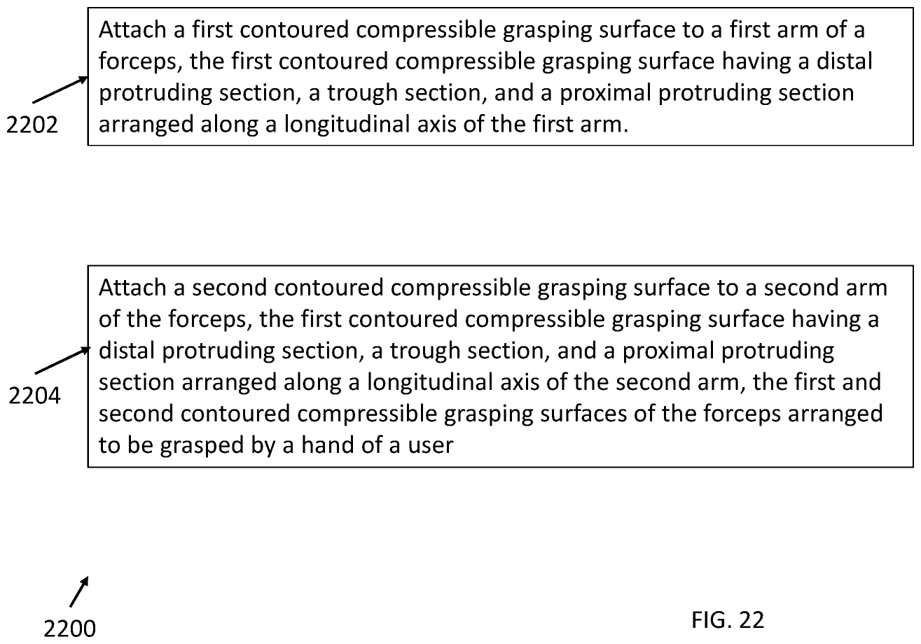

FIG. 22 is a flowchart of a method 2200 for manufacturing a surgical forceps in accordance with various embodiments described herein. The method 2200 includes attaching a first contoured compressible grasping surface to a first arm of a forceps (step 2202). The first contoured compressible grasping surface has a distal protruding section, a trough section, and a proximal protruding section arranged along the longitudinal axis of the first arm. The method 2200 also includes attaching a second contoured compressible grasping surface to a second arm of the forceps (step 2204). The second contoured compressible grasping surface has a distal protruding section, a trough section, and a proximal protruding section arranged along a longitudinal axis of the second arm. The first and second contoured compressible grasping surfaces of the forceps are arranged to be grasped by a hand of a user.

The claims should not be read as limited to the described order or elements unless stated to that effect. All embodiments that came within the scope and spirit of the following claims and equivalents thereto are claimed as the invention.

1. A surgical instrument, comprising:
a handle comprising a contoured compressible grasping surface having a distal protruding section, a trough section, and a proximal protruding section arranged along a longitudinal axis of the handle, at least the trough section including a plurality of spaced protrusions that deform upon application of manual pressure, and
a surgical instrument mounted on a distal end of the handle.

2. The surgical instrument of claim 1, wherein a dimension of the proximal protruding section is greater than a dimension of the distal protruding section.

3. The surgical instrument of claim 2, wherein the dimension of the proximal protruding section is in a range from 12.5-28 mm, or wherein the dimension of the proximal protruding portion is in a range from 14-24 mm, and wherein the dimension of the distal protruding section is in a range from 10-18 mm or wherein the dimension of the distal protruding section is in a range from 12-18 mm.

4. (canceled)

5. (canceled)

6. (canceled)

7. The surgical instrument of claim 2, wherein a dimension of the trough section is less than the dimension of the distal protruding section.

8. The surgical instrument of claim 7, wherein the dimension of the trough section is in a range from 8-18 mm, or the dimension of the trough section is in a range from 9-15 mm.

9. (canceled)

10. The surgical instrument of claim 1, wherein the contoured compressible grasping surface comprises an elastic body that compresses in a range of 1% to 20% of a thickness of the elastic body.

11. The surgical instrument of claim 1, wherein a spacing between spaced protrusions is at least 0.5 mm.

12. The surgical instrument of claim 11, wherein the spaced protrusions comprise raised nobs, hexagonal protrusions, or diamond-shaped protrusions, and wherein a height of the spaced protrusions enables the hand of the user to avoid contacting fluids on the textured or roughened surface of the contoured compressible grasping surface.

13. (canceled)

14. The surgical instrument of claim 11, wherein each spaced protrusion is dome-shaped.

15. The surgical instrument of claim 11, wherein each spaced protrusion has a rounded portion and a flat plateau top.

16. The surgical instrument of claim 1, wherein the compressible grasping surface has a substantially circular and/or elliptical cross-section.

17. The surgical instrument of claim 1, wherein the handle is composed of a rigid polymer, or wherein the handle and the contoured compressible grasping surface are composed of a polymer material is liquid impermeable and sterilizable, or wherein the handle comprises stainless steel on which the compressible material is formed or attached.

18. (canceled)

19. (canceled)

20. The surgical instrument of claim 1 wherein the protrusions have a height and compress by a distance in a range between 6-12% of the height upon application of manual force.

21. A surgical forceps comprising:
a first arm having a first contoured compressible grasping surface; and a second arm attached to the first arm, the second arm having a second contoured compressible grasping surface such that the first and second compressible grasping surfaces of the forceps can be held in a hand of a user, wherein the first contoured compressible surface and the second contoured compressible surface each include a distal protruding section, a trough section, and a larger protruding section extending along a longitudinal axis of each respective arm, and a first tip coupled to the first arm and a second tip coupled to the second arm such that the tips undergo relative movement upon application of manual force to each compressible grasping surface.

22. (canceled)

23. The forceps of claim 21, wherein each compressible grasping surface has a channel or slot to receive a portion of one of the first or second arms.

24. The forceps of claim 21, wherein each compressible grasping surface comprises an elastomeric body further comprising a snap fitting that attaches the elastomeric body to a portion of each corresponding arm.

25. The forceps of claim 21, wherein each compressible grasping surface comprises an elastomeric body attached to a metal forceps portion with an adhesive, and wherein each compressible grasping surface comprises an elastic body that compresses in a range of 1% to 20% of a thickness of the elastic body.

26. (canceled)

27. The forceps of claim 21, wherein each compressible grasping surface further comprises a textured or roughened surface having elevated elements with a spacing between elevated elements of at least 0.5 mm.

28. The forceps of claim 27, wherein the elevated elements comprise raised nobs, domes, hexagonal protrusions, or diamond-shaped protrusions that have a height and that compress a distance between 4-15% of the height upon application of manual grasping force.

29. The forceps of claim 21, wherein the compressible grasping surfaces form a substantially circular and/or elliptical cross-section, and wherein the first and second arms and the first and second tips comprise a rigid polymer and wherein the first compressible grasping surface and the second compressible grasping surface form an unbroken perimeter when the first tip and the second tip are brought together.

30. (canceled)

31. The forceps of claim 21, wherein the first and second arms comprise a polymer material that is sterilizable, or wherein the first arm and second arm comprise stainless steel.

32. (canceled)

33. (canceled)

34. The forceps of claim 21, wherein the first arm and second arm have a smaller width than the respective first contoured compressible surface and the second contoured compressible surface.

35. A surgical forceps comprising: a unitary polymer forceps having a first arm and a second arm; a first contoured grasping surface on the first arm that includes a distal protrusion, a trough and a larger proximal protrusion extending along the first contoured compressible grasping surface; and a second contoured grasping surface on the second arm that includes a distal protrusion, a trough and a larger proximal protrusion extending along the second contoured compressible grasping surface.

36. The forceps of claim 35 wherein the first and second grasping surfaces are compressible and comprise elastomeric bodies that are attached to the polymer arms of the unitary polymer forceps, and wherein the contoured top surfaces comprise raised nobs, domes, hexagonal protrusions, or diamond-shaped protrusions, or raised elements having a height that compresses in a range of 4-15% of the height upon application of a manual grasping force.

37. (canceled)

38. The forceps of claim 35 wherein the contoured top surfaces have a substantially circular cross-section when the first arm and the second arm are compressed to contact, and wherein a first proximal end of the first arm is joined with a second proximal end of the second arm.

39. (canceled)

40. The forceps of claim 38 wherein the first proximal end and the second proximal end define a joint angle greater than 10 degrees.

41. A method for performing a surgical procedure with a surgical instrument, comprising: manually grasping a handle having elevated surface features to provide a contoured compressible grasping surface having a distal protruding section, a trough section, and a proximal protruding section arranged along a longitudinal axis of the handle, the elevated surface features having a height that compress a distance in a range between 4-15% of the height upon application of a manual grasping force.

42. The method of claim 41 wherein the handle has a substantially circular cross-section orthogonal to the longitudinal axis.

43. The method of claim 41 wherein the surgical instrument comprises forceps having a first arm and a second arm joined at a proximal end and wherein distal ends of the arms are compressed together upon application of the manual grasping force.

44. The method of claim 43 wherein the forceps comprise metal arms, each metal arm having a polymer material on each arm to form the compressible grasping surface, and wherein the width of the polymer material is greater than the width of each metal arm.

45. (canceled)

46. The method of claim 41 wherein the surgical instrument comprises a surgical scalpel, or wherein the surgical instrument comprises a dental instrument.

47. (canceled)

48. The method of claim 41 wherein a user can rotate the instrument handle with fingers of a hand that is grasping the instrument, the handle having a substantially circular cross-section through the trough section orthogonal to the longitudinal axis.

49. The method of claim 41 wherein the elevated features are spaced apart concentrically around the handle in at least the trough section, and wherein the elevated features comprise domes, nobs or shaped features for grasping by a user's fingers that frictionally engage the elevated features at varying levels of force.

50. (canceled)
